SEQUENCE LISTING (18 sequences)

SEQ ID NO: 1 - DNA, 1331 bp, Mus musculus
gatatcatgggatggaatgagaagggaaagtaggagcccgagagtgcggtaagacaaggcataaggcgtgtctgacaaattcttcatacacacatttcccctttgcacattcagtctgtataggttatttctataggagaaaaaaaatattcaaattccttgtgcactggtaacaggcatgaaggctcagcaaagccaatacgtgttatgtccagttggagacagtgccagggccaacattccagacttctcagatagaaagtgcgcctgcctgccctgctctgagaatttgaagagagtagttcagttagaattaagaggcagtagagaaaagtcttgggaaatctggttagagatataaatatgagaactggacatggtggtacacacctgtgatctctgtgtttaggagggagaggcagagagatcaggagttcaaggccagcctgagctacttgagacccagtctaaataaataagagatagattacagagtgcctttaactagtacagagaaagaatttgggtttatctgtgtcagttacgctgaaataatttttaagtaataaaatcccttttaataagaaaccttatgaggtcagtatgcacaatgaacttaagagagacccccagctcctgagctgagtgatggggaaggacagccactgcctgtgatgtgtgagtgacgtgcttccaagtgttttaaccactgacgattacatagcctgcacagtcaggagaaaacagccgtattctctgccagttctcttcccttttacaaacagatgagagacacacacagagaatccatttaaagagcggacctttgttctgattaggggcaattttaagtacttaagagttcacacaaagtctagccttcaaaaagaaaacaggttcccaaactagggaggaaacagaatcatttccattttggtgacatttagtgggaagaagctcacagacatttagacgttccaactctttccccactagtggaccaagtatataatatggtatcttttgggcactggtattacaactgttttttaaacaaaagactttccttgtgctttactaaaaacccagacggtgaatcttgaatacaatgcgtggcacccacggcaggcattctattgtgcatagttttgactgacaggagatgacagcatttggctggctgcgcttgctgaggaccctctcctcctgtgtggcgtctgagactgtgatgcaaatgcgcccgcccttttctgggaactcagaacgcctgagtcaggcggcggtggctattaaagcgcctggtcaggctgggctgccgcactgcaaggatg

SEQ ID NO: 2 - DNA, 19 bp, Artificial Sequence
  primer
tgcccacgctgaccatcca

SEQ ID NO: 3 - DNA, 21 bp, Artificial Sequence
  primer
gagacaacgatcggtacccct

SEQ ID NO: 4 - DNA, 21 bp, Artificial Sequence
  primer
acgatgtcttttgttcctggg

SEQ ID NO: 5 - DNA, 21 bp, Artificial Sequence
  primer
tttttttttgtgattcttcca

SEQ ID NO: 6 - DNA, 19 bp, Artificial Sequence
  primer
gccacaaccaaccaggaag

SEQ ID NO: 7 - DNA, 21 bp, Artificial Sequence
  primer
gagctgagtgatggggaagga

SEQ ID NO: 8 - DNA, 20 bp, Artificial Sequence
  primer
ccactgacgattacatagcc

SEQ ID NO: 9 - DNA, 23 bp, Artificial Sequence
  primer
ggacctttgttctgattaggggc

SEQ ID NO: 10 - DNA, 20 bp, Artificial Sequence
  primer
cgcagacatttagacgttcc

SEQ ID NO: 11 - DNA, 21 bp, Artificial Sequence
  primer
gggcactggtattacaactgt

SEQ ID NO: 12 - DNA, 21 bp, Artificial Sequence
  primer
ctcagggcttttatgttgaag

SEQ ID NO: 13 - DNA, 18 bp, Mus musculus
cactagtggaccaagtat

SEQ ID NO: 14 - DNA, 22 bp, Mus musculus
aattatacttggtccactagtg

SEQ ID NO: 15 - DNA, 17 bp, Mus musculus
ttccaactctttcccac

SEQ ID NO: 16 - DNA, 21 bp, Mus musculus
aattgtgggaaagagttgcaa

SEQ ID NO: 17 - DNA, 18 bp, Mus musculus
acagacatttagaggttc

SEQ ID NO: 18 - DNA, 21 bp, Mus musculus
aattgaacgtctaatgtctgt

(19) United States
(12) Patent Application Publication
(10) Pub. No.: US 2004/0062770 A1
(43) Pub. Date: Apr. 1, 2004

(54) Modulators of fgl2 prothrombinase

(76) Inventor: Gary Levy, Thornhill (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/688,962

(22) Filed: Oct. 21, 2003

Related U.S. Application Data
(63) Continuation of application No. 09/926,070, filed on Aug. 24, 2001, now abandoned.
Application No. 09/926,070 is a 371 of international application No. PCT/CA00/00191, filed on Feb. 25, 2000.
(60) Provisional application No. 60/122,109, filed on Feb. 26, 1999.

(51) Int. Cl. A61K 48/00; A61K 39/42

(52) U.S. Cl. 424/161.1; 514/44

(57) ABSTRACT

The inventor has determined that the nucleocapsid protein from a hepatitis virus can induce the prothrombinase, fgl2. The inventor has further developed that LF-A1 also induces fgl-2. This allows for the development of therapeutic methods and compositions for modulating immune coagulation. In particular, inhibitors of the N-protein or gene or LF-AL protein gene, or LF-A1 binding site on the fgl-2 promoter may be useful in inhibiting immune coagulation caused by a virus such as a hepatitis virus.

### FIELD OF THE INVENTION

The invention relates to modulators of the prothrombinase fgl2 and the use of the modulators in controlling immune coagilation.

### BACKGROUND OF THE INVENTION

Activation of the coagulation pathways is an important part of immune and inflammatory reactions and is associated with bacterial and viral infections (e.g. endotoxin shock, viral hepatitis), glomerulonephritis (GN), cancer, a number of gastrointestinal diseases, allograft and xeno graft rejection and spontaneous or stress-triggered fetal loss. Immune coagulation is mediated by a number of coagulants that, when triggered, activate specific ligands resulting in cleavage and activation of coagulation pathways that lead to fibrin deposition. The molecular events leading to expression of immune coagulants involve natural antibodies binding both to antigens on endothelial cells and Fc receptors on macrophages and endothelial cells. An additional mechanism is immune complex-mediated induction of macrophage procoagulants. These events lead to thrombin production which initiates platelet activation and ultimately fibrin deposition.

In 50% of hepatitis patients moderate to severe consumptive coagulopathy, or disseminated intravascular coagulopathy is found associated with fulminant hepatitis. Thrombi formation is observed around necrotic areas (Sinclair et al., 1990 and Lee, W. M., 1993). As a consequence of hepatitis, levels of factors II, V, VII, and X are decreased in the liver, reflecting both consumptive coagulopathy and a decrease in hepatic synthetic function. Also, the levels of thrombin-antithrombin complexes are high and platelet counts are low (Lee, W. M., 1993). These results indicate that the host immune system, including the coagulation pathway, is disrupted as a result of HBV infection. The limited host range of HBV and the difficulty to propagate the virus in tissue culture have hampered the understanding of HBV and hepatitis B.

Murine Coronavirus infection is a model for studying acute and chronic hepatitis of humans. MHV-3 infection in BALB/cJ mice causes fulminant hepatic failure (FHF), which is characterized by macrophage activation and marked production of pro-inflammatory mediators. Especially intriguing is the ability of MHV-3 to induce de-novo synthesis of a unique procoagulant, the fgl2 prothrombinase, encoded by the fgl2 gene located on mouse chromosome 5 (Parr et al., 1995; Ning et al., 1998). Several lines of evidence implicate expression of this gene product in the pathogenesis of fulminant murine hepatitis. Firstly, levels of this prothrombinase activity correlates with the severity of the disease (Levy et al., 1983; Macphee et al., 1985). Secondly, treatment of mice with a neutralizing monoclonal antibody to the MHV-3-induced prothrombinase prevents the lethality associated with MHV-3 infection (Li et al., 1992). Concordant with these observations, expression of fgl-2 prothrombinase in liver accounts for widespread fibrin deposition in hepatic blood vessels and hepatocellular necrosis (Ding et al., 1997).

The MHV genome is a single-stranded non-segmented RNA of approximately 32 Kb (Lai and Stohlman, 1978; Pachuk et al., 1989). The RNA genome contains 7-8 genes, encoding 3-4 structural proteins and 4 nonstructural proteins (Lai and Cavanagh, 1997). An important aspect of MHV biology is the high frequency of RNA-RNA recombination between strains of MHV (Makino et al., 1989; Keck et al., 1997; Makino et al., 1986). RNA recombination may contribute to viral pathogenesis and also provides a useful tool for the study of genetic control of the biologic properties of viruses. Studies using recombinant viruses derived from MHV-JHM and MHV-A59 have demonstrated that the 3′-portion (about 25%) of the viral genome, representing RNA genomic regions encoding for all of the structural proteins, controls biologic properties such as organotropism of the virus, the pattern of the virus-induced central nervous system pathology in mice, plaque morphology, and virus yield in tissue culture (Lavi et al., 1990; Masters and Sturman, 1990).

In view of the foregoing, there is a need to identify and characterize the molecular basis for induction of fgl2 in immune and inflammatory reactions such as in hepatitis caused BY MHV-3.

### SUMMARY OF THE INVENTION

The present inventor has shown that the nucleocapsid protein (N-protein) of mouse hepatitis virus (MHV) induces the transcription of the fgl-2 prothrombinase gene in those strains of MHV which induce fulminant hepatic failure. In particular, the inventor has shown that domain I of the MHV N-protein is required for fgl-2 induction.

Accordingly, the present invention provides a method of inducing immune coagulation comprising administering an effective amount of an nucleocapsid protein or gene or a fragment or analog thereof to an animal in need thereof. In a preferred embodiment, the N-protein comprises domain I of the MHV N-protein sequence or of the corresponding domain of the pathogenic factor of human hepatits virus which is known to induce FGL-2, i.E., Hepatities B and C virus. [Levy, G et al, AASLD, November 1999, Dallas Texas and HEpatology, October 1999]

The present invention also includes a method of reducing or preventing immune coagulation comprising administering an effective amount of an inhibitor to the nucleocapsid protein or gene to an animal in need thereof.

Inhibitors of the N protein include antibodies to the N-protein. Inhibitors of the N-gene include antisense oligonucleotides to the N gene.

The present inventor has also identified that region −372 to −306 of the fgl-2 promoter is the region that is responsive to induction by the N-protein. More, particularly, in a further embodiment, the inventor has further determined that a CIS acting regulatory element, liver factor A1 binding element(LF-A1), found in the fgl2 promoter region at nucleotides −332 to −325 is responsible for fgl-2 induction. The inventor also determined that a probe specific for the LF-A1 binding element binds to the host cell LF-A1 transciption factor protein in the nucleus of a cell induced by strains of mhv known to induce fgl-2, such as, MHV-3 and MHV-A59.

Accordingly, the present invention also includes a method of reducing or preventing immune coagulation comprising administering an effective amount of an inhibitor to the promoter region of fgl2 to an animal in need thereof. Preferably, the inhibitor is an antisense oligonucleotide complimentary to nucleotides −372 to −306 of the fgl-2 gene sequence. [Genbank Accession Number M15761 and PCT/CA98/00475]

The present invention further includes a method of reducing or preventing immune coagulation comprising administering an effective amount of an inhibitor to LF-A1 to an animal in need thereof.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a schematic representation of the oligonucleotide maps of the recombinant viruses.

FIG. 2 is a schematic and nucleic acid sequence of the fgl2 gene.

FIGS. 3A and 3B are bar graphs showing induction of fgl2 in the presence of various viruses.

FIG. 4 shows the expression of fgl2 in macrophages infected with various viruses.

FIG. 5 is a Western blot showing the expression of the N-protein in transfected cells.

FIG. 6 is a bar graph showing the effect of the N-protein on the fgl2 promoter.

FIG. 7A is a bar graph showing transient expression of luciferase activity by deletion constructs of the fgl2 promoter.

FIG. 7B is a schematic of the putative regulatory elements in the fgl2 promoter responsive to N protein.

FIG. 8 is a bar graph illustrating that it is the N-protein and not the I-protein of MHV259 which induces fgl-2 expression.

FIG. 9 a bar graph illustrating that domain I of MHV259 is responsible for inducing fgl-2 expression.

FIG. 10 is a confocus microscope immunofluoresence assay illustrating that N-protein localizes in the nuclesus of infected macrophages.

FIG. 11 is an Electrophoresis Mobility Shift DNA-protein banding assay illustrating that LF-A1 and IE1.2, but not GMSCF oligonucleotide probes bind to a transcription factor protein expressed in nuclear extracts of MHV infected cells.

FIG. 12 is a bar graph illustrating that LFA-1 promoter region of fgl-2 is responsive to the N-protein of MHV.

FIG. 13 is a Western blot illustrating that LFA-1 (HNF-4) is expressed in macrophages.

### DETAILED DESCRIPTION OF THE INVENTION

As hereinbefore mentioned, the present inventors have determined that the nucleocapsid protein of mouse hepatitis virus (MHV) induces the transcription of the prothrombinase, fgl-2. In particular, using a set of parental and recombinant MHV strains, it was demonstrated that the nucleocapsid protein (N) of MHV induces transcription of the fgl-2 prothrombinase gene in those strains of MHV which induce fulminant hepatic failure. Two deletions found at coding sites 111-123 and 1143-1145 of structural domains I and III respectively, of the N gene may account for the important phenotypic differences observed between pathogenic and non-pathogenic strains. The present inventor has also identified that region −372 TO −306 of the fgl-2 promoter is the region that is responsive to induction by the n-protein. The inventor has further determined that a CIS acting regulatory element, liver factor A1 binding element(LF-A1), found in the fgl2 promoter region at nucleotides −332 to −325 is responsible for fgl-2 induction. The inventor also determined that the LF-A1 protein binds to the LF-A1 CIS binding element of the fgl-2 promoter region. [Elizabeth M. Hardon, Monique Frain, Giacomo Paonessa and Riccardo Cortese. Two distinct factors interact with the promoter regions of several liver-specific genes. The Embo Journal Vol. 7 No. 6 pp.1711-1719, 1988]

The above finding by the inventors allows the development of therapeutic methods and compositions for modulating immune coagulation, for example, for inhibiting immune coagulation caused by viral hepatitis infection.

(A) Methods of Inhibiting Immune Coagulation

(1) Inhibiting the N-Protein or N-Gene [New Header]

In one aspect, the present invention provides a method of preventing or reducing immune coagulation caused by a virus comprising administering an effective amount of an inhibitor of a nucleocapsid gene or nucleocapsid protein to an animal in need thereof.

The term “nucleocapsid” gene or protein means a nucleocapsid gene or protein from a virus and is synonymous with “capsid” or “core” gene or protein and is generally abbreviated as N-gene or N-protein. The virus can be any virus, including but not limited to, hepatitis B, C, E or G; murine hepatitis virus; human immunodeficiency virus; T-cell leukemia virus; Hanloan virus and Seoul virus.

The term “effective amount” as used herein means an amount effective and at dosages and for periods of time necessary to achieve the desired result.

The term “animal” means any member of the animal kingdom including all mammals, preferably humans.

The term “fgl-2” means any member of the fgl-2 family, including murine fgl-2 (mfgl-2) and human fgl-2(hfgl-2).

In one embodiment, the present invention provides a method of preventing or treating hepatitis comprising administering an effective amount of an inhibitor of a nucleocapsid gene or nucleocapsid protein to an animal in need thereof, preferably, the inhibitor binds to domain I of the hepatitis virus which is capable of inducing fgl-2.

(i) Antibodies

Polyclonal and monoclonal antibodies that bind to and neutralize the N-protein can be used to inhibit the N-protein.

Accordingly, the present invention provides a method of reducing or preventing immune coagulation caused by a virus comprising administering an effective amount of an antibody to an N-protein, or more preferably to domain I of the N-protein, to an animal in need thereof.

Antibodies that bind an N-protein can be prepared using techniques known in the art such as those described by Kohler and Mistein, Nature 256, 495 (1975) and in U.S. Pat. Nos. RE 32,011, 4,902,614, 4,543,439, and 4,411,993 which are incorporated herein by reference. (See also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference).

Within the context of the present invention, antibodies are understood to include monoclonal antibodies, polyclonal antibodies, antibody fragments (e.g., Fab, and F(ab′)₂) and recombinantly produced binding partners. Antibodies are understood to be reactive against the protein encoded by the nucleic acid molecule of the invention if they bind to N-protein with an affinity of greater than or equal to 10⁻⁶ M. As will be appreciated by one of ordinary skill in the art, antibodies may be developed which not only bind to the protein, but which bind to a regulator of the protein, and which also block the biological activity of the protein.

Polyclonal antibodies may be readily generated by one of ordinary skill in the art from a variety of warm-blooded animals such as horses, cows, various fowl, rabbits, mice, or rats. Briefly, a N-protein of the invention or portions thereof, may be used to immunize an animal. An animal may be immunized through intraperitoneal, intramuscular, intraocular, or subcutaneous injections, in conjunction with an adjuvant such as Freund's complete or incomplete adjuvant. Following several booster immunizations, samples of serum are collected and tested for reactivity to the protein. Particularly preferred polyclonal antisera will give a signal on one of these assays that is at least three times greater than background. Once the titer of the animal has reached a plateau in terms of its reactivity to the protein, larger quantities of antisera may be readily obtained either by weekly bleedings, or by exsanguinating the animal.

Monoclonal antibodies may also be readily generated using conventional techniques as described herein. Generally, hybridoma cell lines are prepared by a process involving the fusion under appropriate conditions of an immortalizing cell line and spleen cells from an animal appropriately immunized to produce the desired antibody. Immortalizing cell lines may be murine in origin however, cell lines of other mammalian species may be employed including those of rat, bovine, canine, human origin, and the like. The immortalizing cell lines are most often of tumor origin, particularly myeloma cells but may also include normal cells transformed with, for example, Epstein Barr Virus. Any immortalizing cell may be used to prepare the hybridomas of the present invention.

Antibody producing cells may be employed as fusion partners such as spleen cells or peripheral blood lymphocytes. The aminal from which the cells are to be derived may be immunized at intervals with peptides derived from N-protein.

The immortalizing cells and lymphoid cells may be fused to form hybridomas according to standard and well-known techniques employing polyethylene glycol as a fusing agent. Alternatively, fusion may be accomplished by electrofusion.

Hybridomas are screened for appropriate monoclonal antibody secretion by assaying the supernatant or protein purified from the ascites for reactivity using the method described herein. The hybridomas are screened for antibodies which have the desired properties e.g. neutralize the N-protein and inhibit the prothrombinase activity of Fgl2.

The monoclonal antibodies produced by the hybridoma cell lines of the invention are also part of the present invention. It is understood that immunoglobulins may exist in acidic, basic, or neutral form depending on their amino acid composition and environment, and they may be found in association with other molecules such as saccharides or lipids. The monoclonal antibodies produced by hybridoma cell lines of the invention may be directed against one or more of epitopes of N-protein. Any characteristic epitope associated with N-protein may provide the requisite antigenic determinant. It is contemplated that monoclonal antibodies produced by the hybridoma cell lines fall within the scope of the present invention so long as they remain capable of selectively reacting with peptides from N-protein.

The antigens recognized by the monoclonal antibodies described herein are also a part of the present invention. An antigen recognized by a monoclonal antibody produced by a hybridoma cell line of the invention, may be localized to specific cells and tissues using conventional immunocytochemistry methods. Cryostat sections may be incubated with a monoclonal antibody of the invention and processed by the avidin-biotin-peroxidase technique (ABC Vectastain). This will determine which class of cells express an antigen of N-protein.

The present invention includes recombinant or chimeric antibody molecules. Such antibodies or binding partners may be constructed utilizing recombinant DNA techniques to incorporate the variable regions of a gene which encodes a specifically binding antibody. Within one embodiment, the genes which encode the variable region from a hybridoma producing a monoclonal antibody of interest are amplified using nucleotide primers for the variable region. These primers may be synthesized by one of ordinary skill in the art, or may be purchased from commercially available sources. Primers for mouse and human variable regions including, among others, primers for V_{Ha}, V_{Hb}, V_{Hc}, V_{Hd}, C_{H1}, V_{L} and C_{L} regions are available from Stratacyte (La Jolla, Calif.). These primers may be utilized to amplify heavy or light chain variable regions, which may then be inserted into vectors such as ImmunoZAP H or ImmunoZAP L (Stratacyte), respectively. These vectors may then be introduced into E. coli for expression. Utilizing these techniques, large amounts of a single-chain protein containing a fusion of the VH and VL domains may be produced (See Bird et al., Science 242:423-426, 1988). In addition, such techniques may be utilized to produce a “human” antibody, without altering the binding specificity of the antibody.

(ii) Antisense Molecules

Antisense oligonucleotides that are complimentary to a nucleic acid sequence from a N-protein gene can also be used in the methods of the present invention to inhibit N-gene activity.

Accordingly, the present invention provides a method of preventing or reducing immune coagulation caused by a virus comprising administering an effective amount of an antisense oligonucleotide that is complimentary to a nucleic acid sequence from an N-gene to an animal in need thereof.

The term “antisense oligonucleotide” as used herein means a nucleotide sequence that is complimentary to its target.

The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates, and phosphorodithioates. In an embodiment of the invention there are phosphorothioate bonds links between the four to six 3′-terminus bases. In another embodiment phosphorothioate bonds link all the nucleotides.

The antisense oligonucleotides of the invention may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogues have been shown to be resistant to degradation by enzymes and to have extended lives in vivo and in vitro. PNAs also bind stronger to a complimentary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Oligonucleotides may also contain groups such as reporter groups, a group for improving the pharmacokinetic properties of an oligonucleotide, or a group for improving the pharmacodynamic properties of an antisense oligonucleotide. Antisense oligonucleotides may also have sugar mimetics.

The antisense nucleic acid molecules may be constructed using chemical synthesis and enzymatic ligation reactions using procedures known in the art. The antisense nucleic acid molecules of the invention or a fragment thereof, may be chemically synthesized using naturally occurring nucleotides or variously modified nucleotides designed to increase the biological stability of the molecules or to increase the physical stability of the duplex formed with. mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The antisense oligonucleotides may be introduced into tissues or cells using techniques in the art including vectors (retroviral vectors, adenoviral vectors and DNA virus vectors) or physical techniques such as microinjection. The antisense oligonucleotides may be directly administered in vivo or may be used to transfect cells in vitro which are then administered in vivo. In one embodiment, the antisense oligonucleotide may be delivered to macrophages and/or endothelial cells in a liposome formulation.

(iii) Other N-Protein Inhibitors

In addition to antibodies and antisense oligonucleotides, other substances that inhibit N-protein may be isolated. N-protein binding peptides may be isolated by assaying a sample for peptides that bind to the N-protein or domain I of the N-protein. Any assay system or testing method that detects protein-protein interactions may be used including co-immunoprecipitation, crosslinking and co-purification through gradients or chromatographic columns may be used. Biological samples and commercially available libraries may be tested for N-protein binding peptides. For example, labelled N-protein may be used to probe phage display libraries. In addition, antibodies prepared to the peptides of the invention may be used to isolate other peptides with N-protein binding affinity. For example, labelled antibodies may be used to probe phage display libraries or biological samples.

Additionally, a DNA sequence encoding a N-protein may be used to probe biological samples or libraries for nucleic acids that encode N-protein-binding proteins.

(2) Inhibiting LF-A1

In another aspect, the present invention provides a method of reducing or preventing immune coagulation comprising administering an effective amount of an inhibitor to LF-A1 to an animal in need thereof.

Inhibitors of LF-A1 include antibodies and antisense molecules to LF-A1 that may be prepared using standard techniques as described above for the N-protein and gene. In addition other molecules that inhibit LF-A1 may be isolated using the methods described above for isolating inhibitors of the N-protein or gene.

(3) Inhibiting FGL-2 Promoter Region

In a further aspect, the present invention provides a method of reducing or preventing immune coagulation comprising administering an effective amount of an inhibitor to the promoter region of fgl-2 to an animal in need thereof. Preferably, the inhibitor is an antisense oligonucleotide complimentary to nucleotides −372 TO −306 , or preferably −332 to −325, of the fgl-2 Gene sequence. (Genbank Accession Number m15761 and PCT/CA98/00475).

(B) Methods of Inducing Immune Coagulation

In an alternate embodiment, the present invention includes methods of inducing immune coagulation by administering an N-protein or gene. Methods that induce immune coagulation may be useful in treating conditions which require an increase in coagulant activity. Such methods can also be used to induce fetal loss.

Accordingly, the present invention provides a method of inducing immune coagulation comprising administering an effective amount of an N-protein or gene or a fragment or analog therefof to an animal in need thereof.

As used herein, a fragment of a N-protein or gene means any portion of the full length gene or protein that is sufficient to induce fgl-2 transcription or activity. Preferably, the fragment comprises domain I of the N-gene which includes nucleotides 400-500 of the N-gene sequence. [Genbank Accession number M35156]

As used herein, an analog (or mimetic) of the N-gene or protein means any molecule that is functionally similiar to the N-protein or gene in that it can also induce fgl-2 transcription or activity. Analogs or mimetics of the n-protein or gene that may be useful in the present invention may be identified using methods known in the art.

In a preferred embodiment, the n-protein analog is a structural as well as functional analog which includes any peptide having an amino acid residue sequence substantially identical to the N-protein in which one or more residues have been conservatively substituted with a functionally similar residue and which displays the ability to mimic the N-protein in inducing fgl-2 activity. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue in place of a non-derivatized residue provided that such polypeptide displays the requisite activity.

Structural analogs of the N-protein may be identified by probing biological samples and commercially available peptide libraries with an antibody that binds to the N-protein as described herein above. For example, labelled antibodies to N-protein may be used to probe phage display libraries. Any assay system or testing method that detects protein-protein interactions may be used to measure the binding of the antibody to a peptide in the sample including co-immunoprecipitation, crosslinking and co-purification through gradients or chromatographic columns may be used.

The structure of the N-Protein may also be used to identify lead compounds for drug development for use in methods of inducing immune coagulation. A comparison of the structures of peptides similar in sequence to the N-protein, but differing in the biological activities they elicit in target fgl-2 molecules can provide information about the structure-activity relationship of the induction of fgl-2. Information obtained from the examination of structure-activity relationships can be used to design either modified peptides, or other small molecules or lead compounds which can be tested for the induction of fgl-2. The structure of thepeptides identified can be readily determined by a number of methods such as NMR and X-ray crystallography.

Fragments and analogs and structural analogs for the LF-A1 protein shall have a corresponding meaning.

(C) Compositions

The antibodies, antisense oligonucleotides or other inhibitors of N-protein identified using the methods described herein as well as the N-protein and nucleic acid sequences including fragments and analogs, may be incorporated into a pharmaceutical composition containing the substance, alone or together with other active substances.

In one aspect, the present invention provides a composition for use in inhibiting procoagulant activity in an animal comprising (a) an antibody specific for a N-protein; (b) antisense nucleic acid molecules complimentary to N-protein; or (c) an inhibitor identified using the methods as described above in admixture with a suitable diluent or carrier.

In another aspect, the present invention provides a composition for use in inducing procoagulant activity in an animal comprising a nucleic acid sequence encoding N-protein or an N-protein or a fragment or analog of the n-protein or the nucleic acid encoding the N-protein in admixture with a suitable diluent or carrier.

In another embodiment the present invention provides a composition for use in inducing procoagulant activity comprising an effective amount of LF-A1 protein or fragment or analog thereof in admixture with a suitable diluent or carrier.

In another aspect the present invention provides a composition for suppressing or inhibiting procoagulant activity comprising an effective amount of an inhibotr of LF-A1 in admixture with a suitable diluent or carrier. Such an inhibitor can include but is not limited to an antibody to LF-A1 or to a molecule which blocks the binding of LF-A1 to the LF-A1 CIS binding element of the fgl-2 promoter, such as an anti-sense oligonucleotide to the LF-A1 binding element of the promoter region of fgl-2.

Such pharmaceutical compositions can be for oral, topical, rectal, parenteral, local, inhalant or subcutaneous, intradermal, intramuscular, intathecal, vaginal, transperitoneal, placental and intracerebral use. They can be in liquid, solid or semisolid form, for example pills, tablets, creams, gelatin capsules, capsules, suppositories, soft gelatin capsules, gels, membranes, tubelets, solutions or suspensions.

The pharmaceutical compositions of the invention can be intended for administration to humans or animals. Dosages to be administered depend on individual needs, on the desired effect and on the chosen route of administration.

The pharmaceutical compositions can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

On this basis, the pharmaceutical compositions include, albeit not exclusively, the active compound or substance in association with one or more pharmaceutically acceptable vehicles or diluents, and contained in buffered solutions with a suitable pH and iso-osmotic with the physiological fluids. The pharmaceutical compositions may additionally contain other agents such as adjuvants to enhance immune responsiveness.

The antisense nucleic acid molecules of the invention may be used in gene therapy to inhibit immune procoagulant activity in animal with a viral infection. Recombinant molecules comprising an antisense sequence or oligonucleotide fragment thereof, may be directly introduced into cells or tissues in vivo using delivery vehicles such as retroviral vectors, adenoviral vectors and DNA virus vectors. They may also be introduced into cells in vivo using physical techniques such as microinjection and electroporation or chemical methods such as coprecipitation and incorporation of DNA into liposomes. Recombinant molecules may also be delivered in the form of an aerosol or by lavage. The antisense nucleic acid molecules of the invention may also be applied extracellularly such as by direct injection into cells.

(D) Vaccines

The present invention also contemplates a vaccine against a virus causing immune coagulation comprising an amount of an N-protein or peptide which is effective to induce an immune response against N-protein.

In one embodiment, the present invention provides a vaccine for treating or preventing viral hepatitis comprising an effective amount of an N-protein or peptide in admixture with a suitable diluent or carrier.

The vaccine may be a multivalent vaccine and additionally contain immunogens related to other diseases in a prophylactically or therapeutically effective manner.

The vaccine may also comprise an immunologically acceptable carrier such as aqueous diluents, suspending aids, buffers, excipients, and one or more adjuvants known in the art. Examples of adjuvants include the lipid A portion of gram negative bacteria undotoxin, trehalose dimycolate of mycobacteria, the phospholipid lysoleathin, dimethyl dictadecyl ammonium bromide (DDA), linear polyoxypropylene-polyoxyethylene (POP-POE) block polymers and liposomes. The vaccine may also contain cytokines that can enhance the immune response including GM-CSF, IL-2, IL-12, TNF and IFNγ. The vaccine may also contain preservatives such as sodium azide, thimersol, beta propiolactone, and binary ethyleneimine.

The vaccines of the invention can be intended for administration to animals, including mammals, avian species, and fish; preferably humans and various other mammals, including bovines, equines, and swine.

The vaccines of the invention may be administered in a convenient manner, such as intravenously, intramuscularly, subcutaneously, intraperitoneally, intranasally or orally. The dosage will depend on the nature of the disease, on the desired effect and on the chosen route of administration, and other factors known to persons skilled in the art.

A vaccine prepared using the methods described herein may be tested in in vivo animal systems to confirm their efficacy in the prophylaxis or active immunization and treatment of the relevant disease and to determine appropriate dosages and routes of administration.

The present invention also includes the use of the antibodies that bind the N-proteins and portions thereof of the invention as a means of passive immunization.

The present invention also includes DNA immunization with an N-protein gene or portion thereof.

The following non-limiting examples are illustrative of the present invention:

### EXAMPLES

### Example 1

Induction of FGL2 by N-Gene of MHV-3

Materials and Methods

Mice

Female Balb/cJ mice, 6-8 weeks of age, from Charles River Laboratories (St. Constant, Quebec) were kept in microisolated cages and housed in the animal facilities at the Toronto Hospital, and fed a standard Lab chow diet and water ad libitum.

Virus

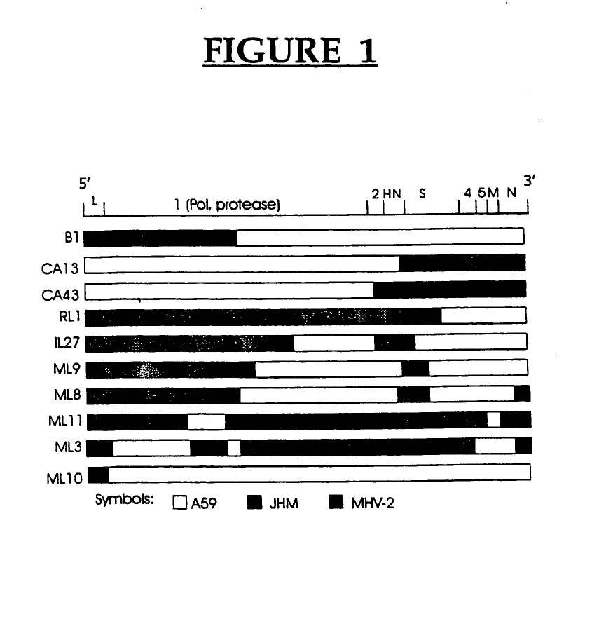

MHV-3 was obtained from American Type Culture Collection [(ATCC), Rockville, Md.] and plaque purified on monolayers of DBT cells. Parental viruses A59, JHM, MHV-2 and 2 sets of recombinant viruses have been described previously (Lai, 1992). The schematic representations of oligonucleotide maps of the recombinant viruses are presented in FIG. 1 (Lai, 1992). To ensure that the recombinant virus strains are clonal, ML3 and ML11 were plaque purified 3 times in 17Cl-1 cells. The purified strains were used for creating N gene expression constructs and transfection experiments.

Cells

Peritoneal macrophages were harvested from Balb/cJ mice 4 days after intraperitoneal administration of 1.5 ml of 3% thioglycolate (Difco Laboratories, Detroit, Mich.) as previously described (Ding et al., 1997). Macrophages were resuspended in RPMI 1640 (ICN Biomedicals Inc., Costa Mesa, Calif.) supplemented with 2 mM glutamine (Sigma Chemical Co., St. Louis, Mo.) and 2% heat-inactivated fetal calf serum (Flow Laboratories, Mississauga, Ontario, Canada). Macrophages were greater than 95% in purity as determined by morphology and non-specific esterase stain. Viability exceeded 95% by trypan blue exclusion. Chinese hamster ovary cell line (CHO cells) were from ATCC.

PCA

MHV and recombinant virus-infected macrophages, at a multiplicity of infection (MOI) of 2.5, were incubated for 8h in RPMI 1640 supplemented with 10% Fetal Bovine Serum and 200 mM glutamine. Mock-infected macrophages and MHV-3-infected macrophages represented negative and positive controls, respectively. Macrophages were evaluated for functional PCA in a one-stage clotting assay, as previously described (Levy et al., 1981). Following incubation, samples to be assayed for PCA were washed three times with unsupplemented RPMI 1640 and resuspended at a concentration of 106/ml. Samples were assayed for the ability to shorten the spontaneous clotting time of normal citrated human platelet-poor plasma. Milliunits of PCA were assigned by reference to a standard curve generated with serial log dilutions of a standard rabbit brain thromboplastin (Dade Division, American Hospital Supply Co., Miami, Fla.).

RT-PCR

Expression of fgl2 was detected by RT-PCR. Freshly isolated macrophages, at a multiplicity of infection (MOI) of 2.5, were infected with different strains of viruses for 6 hours. 1×107 macrophages were pelleted in 1.5 ml Eppendorf tube and total cellular RNA was isolated by 8M acid-guanidium hydrochloride extraction in a modified procedure as previously described (Evans and Kamdar, 1990). The quantity and quality of RNA was examined by spectrophotometry and on a 1% analytical agarose gel containing formaldehyde. RNA (5 μg) was reverse transcribed using Moloney Murine Leukemia Virus Reverse Transcriptase (M-MLVRT) in 20 μl reactions, as recommended by manufacturer. PCR was then performed in 50 μl reactions using 1 μl portion of cDNA and the primers fgl2-318 (TGC CCA CGC TGA CCA TCC A) corresponding to nucleotide 318 \overline{n} 336 of Balb/c fgl2 cDNA (M 15761 ) and FGL2-1224 (GAG ACA ACG ATC GGT ACC CCT) corresponding to nucleotides 1224 -1244 of Balb/c fgl2 cDNA. (M16238), which yield a 906 bp band in 1% agarose DNA gel. Amplification products were not obtained when reverse transcriptase was omitted (data not shown). RT-PCR for GAPDH was also set up as an internal control to assess the quality of first strand synthesis.

Creation of N Gene and fgl2 Promoter Constructs

Restriction enzymes used to create constructs were obtained from GIBCO BRL, Life Technologies, USA. All plasmids were purified using Qiagen Maxiprep Kits, and grown in DH5 E. coli bacteria (GIBCO BRL).

MHV-2 and A59 are different sizes. The entire N gene coding regions and 3′ UTRs of MHV-A59, MHV-2, ML3 and ML11 were amplified by RT-PCR. RNA was originally extracted from infected macrophages. The sense primer ACG ATG TCT TTT GTT CCT GGG was phosphorylated chemically at position 1 to achieve directed insertion and ligation of PCR products to its vector; the anti sense primer at position 1654 TTT TTT TTT GTG ATT CTT CCA had a poly T group to match the poly A tail at the 3′ end of nucleocapsid genomic RNA. The N gene fragments were subcloned into the 5.0 Kb expression vector pCR 3.1 (Invitrogen), under the control of the CMV promoter and bovine growth hormone 3′-processing signals. External restriction endonuclease Hind III and Pst I and internal restriction endonuclease Eco RI, Eco RV were used to analysis the size and orientation of N gene insert in recombinant plasmid constructs.

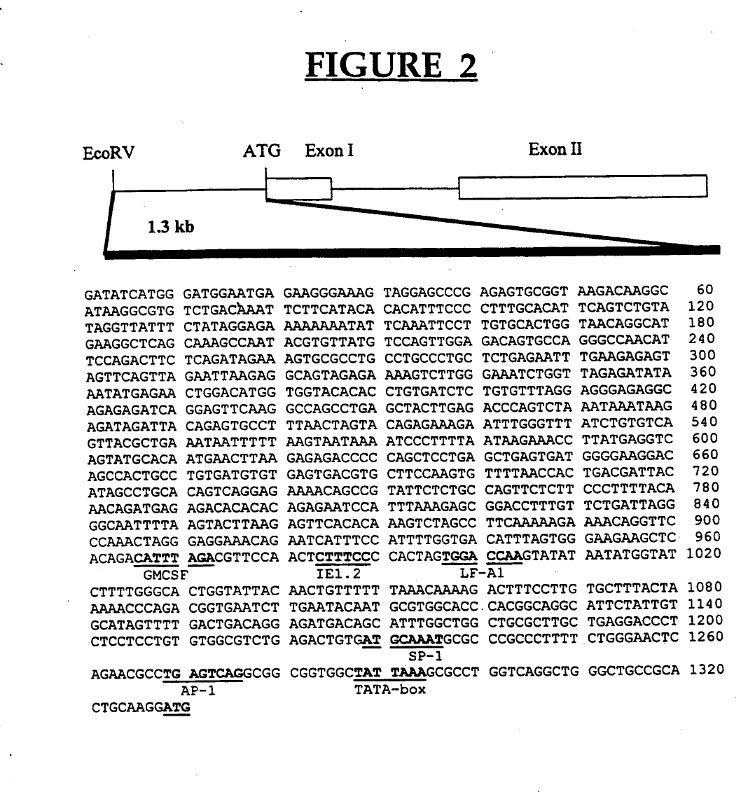

Luciferase Reporter Constructs: A 1.3 kb DNA fragment flanking the 5′ end of mouse fgl2 was released by restriction digestion with EcoRV and Sal I from a subclone pBluescript-m166 (pm166) of mouse genomic P1 plasmid (Genome System Inc.) which contains the entire mouse fgl2 gene. This fragment was sequenced by cycle sequencing on an automated DNA sequencer (Model 377, Applied Biosystems) using dideoxy dye terminator chemistry. This sequence has been deposited into Genbank with the Accession number AF025817 (FIG. 2) (Koyama et al., 1987). This 1.3 kb fragment was inserted into Sma I and Xho I sites of the pGL2-basic luciferase reporter vector (Promega) to form pfgl2(-1328)LUC. 5′ deletion constructs of fgl2 promoter were made by first amplifying the specific fragment using pm166 as template and then cloned into PCR 2.1 cloning vector (Strategene) and resubcloned into pGL2-basic plasmid at Xho I and Hind III sites. The reverse primer (GCC ACA ACC AAC CAG GAA G) was used to make all deletion constructs by PCR amplification. The upstream primers used were: GAG CTG AGT GAT GGG GAA GGA for pfgl2(-693)LUC, CCA CTG ACG ATT ACA TAG CC for pfgl2(-625)LUC, GGA CCT TTG TTC TGA TTA GGG GC for pfgl2(-511)LUC, CGC AGA CAT TTA GAC GTT CC for pfgl2(-372)LUC, and GGG CAC TGG TAT TAC AAC TGT for pfgl2(-306)LUC. All promoter-luciferase report constructs were sequenced to confirm the orientation and to verify the sequence. Positive control, pGL2 Control plasmid with SV-40 promoter, and RSV β-gal vector were from Promega. A 2 Kb tissue factor promoter construct pTF(-2 Kb)LUC was a kind gift of Dr. Nigel Mackman (Mackman et al., 1990).

N Gene Sequence of Different Virus Strains

The N gene sequence of multiple clones for each strain of virus was determined using primer-directed strategies by cycle sequencing on an automated DNA sequencer, using the ABI PRISMTM dRhodamine Terminator Cycle Sequencing Reading Reaction Kit (Model 377, PE Applied Biosystems). The T7 primer and pCR3.1 reverse primer were used for 5′ to 3′ and 3′ to 5′ sequence, respectively. A new primer CTC AGG GCT TTT ATG TTG AAG (MHV-ND557) at position 557 was also designed based on the outcome of sequencing and the published cDNA sequence of MHV-A59 to complete the sequencing. Extension products were purified by ethanol/sodium acetate precipitation. Samples were subjected to electrophoresis on the ABI PRISM 310. The sequence was analyzed using the DNAsis for windows, sequence analysis software (Hitachi Software Engineering America Ltd, San Bruno, Calif., USA).

Transfection

CHO were cultured in 6-well plates until 50-80% confluence. 1 μg of N gene construct DNA, 0.5 μg of fgl2 promoter construct DNA and 0.25 μg of beta-gal DNA (as a marker for transfection efficiency by beta-gal assay) in 100 μl of OPTI-DMEM medium were mixed by vortexing with 3.5 μl of lipofectAMINETM (2 μg/μl) in 100 μl of OPTI-DMEM medium. After incubation of the mixture at room temperature for 30 min, 1.8 ml of OPTI-DMEM medium were added to bring up the volume to 2 ml. One ml of this mixture were distributed into one of the duplicated wells with CHO cells and transfection were proposed at 37° C. with 5% CO₂ for 44-48 hrs. Cells were harvested in lysis buffer and freeze-thawed 3 times in liquid N2. Aliquots of supernatants were assayed for beta-galactosidase and luciferase activity.

Western Blot

At 48 hrs post transfection with N gene and fgl2 (−1.3 Kb/+9) promoter constructs, cultures of 1×106 of CHO cells were collected and lysed in 100 μl of Western blot lysis buffer with protease inhibitor. Twenty (20) μl of lysate were resolved by SDSPAGE and then transferred to a nitrocellulose membrane. After blocking in 4% milk-PBS for 1 hr, membrane was probed with a monoclonal antibody against the N protein at 4° C. overnight, followed by washing in 2% milk-PBS Tween a total of 5 times. The membrane was then incubated with goat anti mouse IgG labeled with horseradish peroxidase for 1 hr and washed in 2% milk-PBS Tween 5 times. Substrates luminol and enhancer were added and incubated for 1 min. The membrane was then exposed to Kodak XAR-5 film with intensifying screens for 10 minutes.

Statistical Analysis

Data are expressed as mean+standard deviation (SD) where applicable. Student's t test for unpaired samples (two tailed) was used to analyze the data.

(a) Expression of Balb/c Macrophage Functional PCA Induced by Parental Viruses and Their Recombinants.

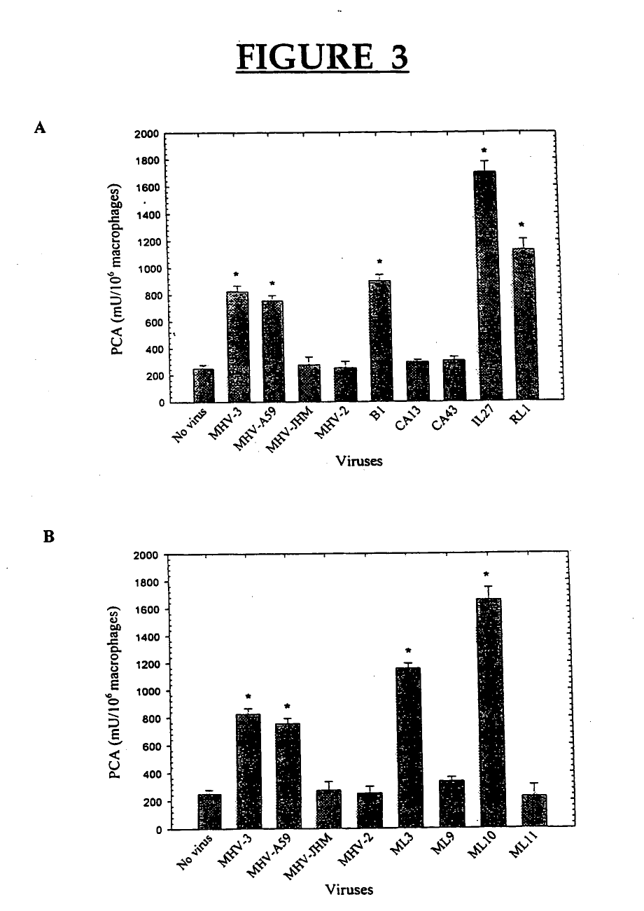

Studies were undertaken using parental A59, JHM and MHV-2 strains and 2 sets of recombinant viruses between them (A59×JHM, A59×MHV-2) (FIG. 1). MHV-A59 infection of macrophages resulted in a marked elevation of functional procoagulant activity (PCA), similar to what was reported previously for MHV-3 (Parr et al., 1995 and Ding et al., 1997) whereas JHM and MHV-2 failed to induce PCA (FIG. 3A). A59×JHM-derived recombinants B1, RL1 and IL27, in which the 3′-portion of the genome is derived from MHV-A59, induced high level of functional PCA. In contrast, CA13 and CA43, two recombinants in which the 3′-portion of the genome is derived from MHV-JHM, did not induce PCA, suggesting that the 3′-portion of the MHV-A59 genome may contain a viral genetic determinant needed for induction of functional PCA, which is lacking in the corresponding region of MHV-JHM. To better delineate the candidate genes required for induction of fgl2, we next studied a set of recombinants derived from MHV-A59×MHV-2, which have multiple crossovers in a single genome, particularly within the 3′ portion of the genome. Recombinants ML3 and ML10 induced functional PCA, while ML11 did not (FIG. 3B). The differences between recombinants ML3 and ML11 in the 3′-portion the genome maps within the 5′-end of the N gene. Therefore, the ability of recombinants to induce functional PCA parallels the presence or absence of MHV-A59 sequences representing the N gene, strongly suggesting that the MHV-A59 N gene is responsible for activation of fgl2.

(b) Induction of fgl2 Transcription by Parental and Recombinant Viruses.

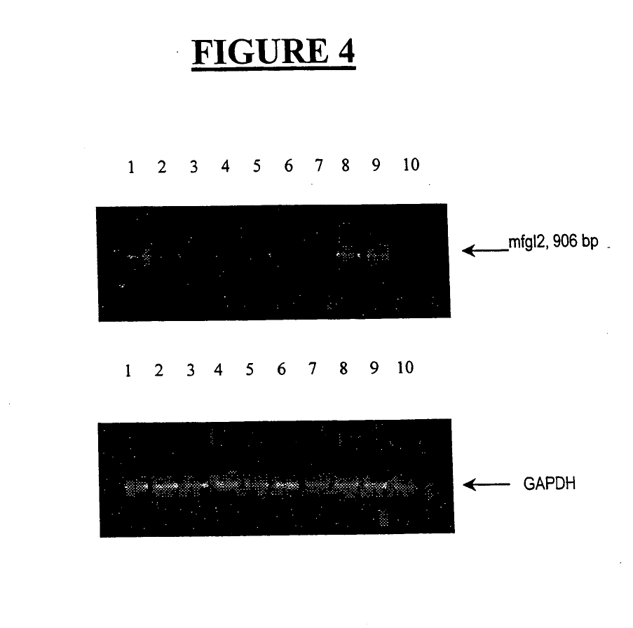

RT-PCR analysis demonstrated that the presence or absence of detectable fgl2 mRNA in macrophages infected with various parental or recombinant viruses correlated with the results of assays for functional fgl2 prothrombinase (FIG. 4).

(c) Sequence Comparison of N-Genes

The entire N gene sequence was determined for MHV-A59, MHV-2, and the recombinant viruses ML3 and ML11, as described in Materials and Methods (sequence data not shown).

A comparison of different N gene sequences is shown in Table 1. Compared to the published sequence of A59 in GeneBank (M35156), the sequence of MHV-A59 used in this study had two point mutations at nt 441 (T→A) and nt 1613 (T→C); ML3 had three point mutations at nt 405 (C→A), nt 441 (T→A), nt 1613 (T→C). Sequence differences between the N genes of MHV-A59 and MHV-2 (AF061835) were mainly within two regions corresponding to nt 400-500 (domain I) and nt 1100-1200 (domain III) of the MHV-A59 sequence. In addition, MHV-2 had a 12 nt-deletion at nt 111-123 and a 3-nt insertion at nt 1143-1145 when compared with MHV-A59. In contrast, the 3′-untranslated regions (UTRs) following the N gene are completely identical between MHV-A59 and MHV-2.

(d) Effect of N Protein on fgl2 Promoter Activity

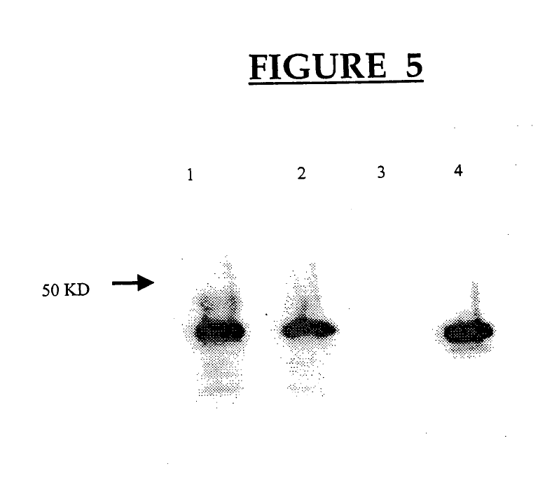

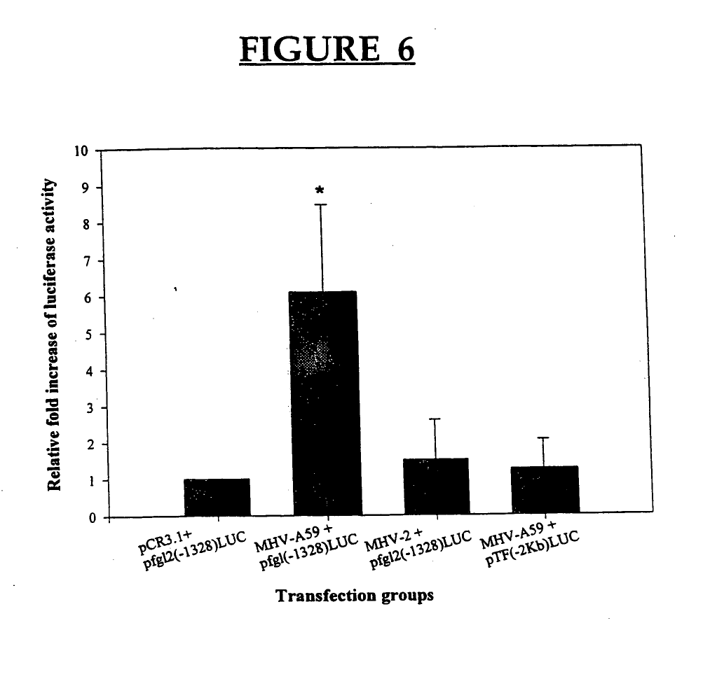

To establish that the N gene of MHV is responsible for the induction of fgl2 gene, co-transfection of CHO cells with the N gene under CMV promoter and fgl2 promoter-luciferase reporter constructs were performed. The expression of the N protein was confirmed by Western blotting (FIG. 5). CHO cells co-transfected with the N gene construct from A59 and a murine fgl-2 promoter/reporter luciferase construct showed a 6-fold increase in luciferase activity compared to cells transfected only with the fgl-2 promoter/reporter luciferase construct (FIG. 6). In contrast, MHV-2 N protein expression did not enhance fgl-2 expression. To confirm the specificity of the effect of N protein on fgl2 promoter activity, pTF(−2Kb)LUC and a pGL2-control vector under SV 40 promoter were cotransfected respectively with the MHV-A59 N gene construct. There was no significant increase in luciferase expression when these 2 constructs were co-transfected respectively with or without the MHV-A59 N gene construct (FIG. 6).

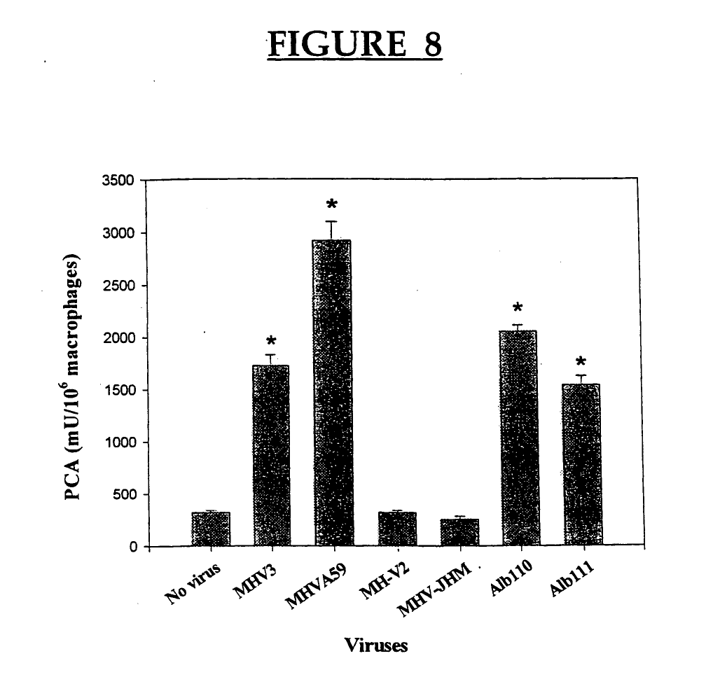

This was further evidenced by studying the effect of N- and I-protein [Fischer et al., Journal of Virology, February, 1997, p. 996-1003, vol 71, No. 2] mutations of domain I of MHV on expression of Balb/cJ macrophage functional PCA. Macrophages from Balb/cJ mice were infected with MHV-3, MHV-A59, N-mutants: MHV-2 and MHV-JHM, and I-mutants: MHV-ALB110 and MHV-ALB111 at a M.O.I. of 2D.5 for 8-10 hrs and harvested for measurement of pca activity. The results are shown in FIG. 8, where values represent the mean ±S.D. of three separate experiments done in triplicate. * represents P<0.01 compared with unstimulated macrophages. The results indicate that MHV-3, MHV-A59 and the I-protein mutations induced Balb/cJ macrophage functional PCA, while MHV-2 does not.

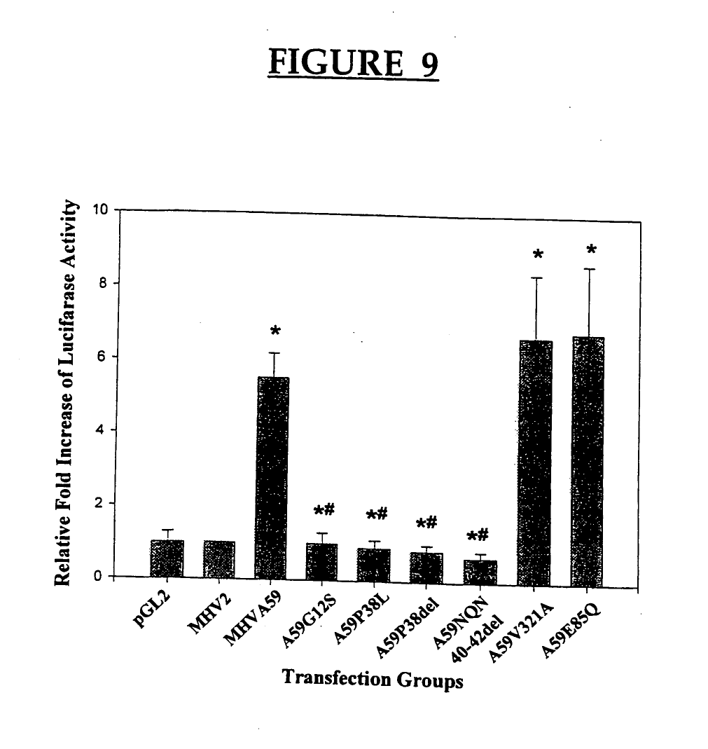

To establish which portion of the n-protein was responsible for fgl-2 induction, N gene constructs from MHV-A59 and MHV-2 and a series of N gene mutants from MHV-A59 domain I (A59G12S, A59P38L, A59P38DEL, A59NQN 40-42DEL) and domain III (A50V321A AND A59E85Q) were cotransfected with the wild type fgl2 promoter-LUC respectively into cho cells. The results are shown in FIG. 9, where relative luciferase activity is expressed in fold increase compared with cho cells cotransfected with n contruct from MHV-2. PGL@-basic vector was used as a negative control. Values represent the mean ±S.D. of fiveseparate experiments done in triplicate. I represents P<0.01 compared with cells cotransfected with MHV-2 N construct. # Represents P<0.01 compared with cells cotransfected with n gene construct from wild type MHV-A59. As can be seen by FIG. 9, mutations in structural domain I did not induce fgl2-promoter-luciferase activity, which suggests that it is domain I which is responsible for fgl-2 induction.

(e) Mapping of the fgl2 Promoter.

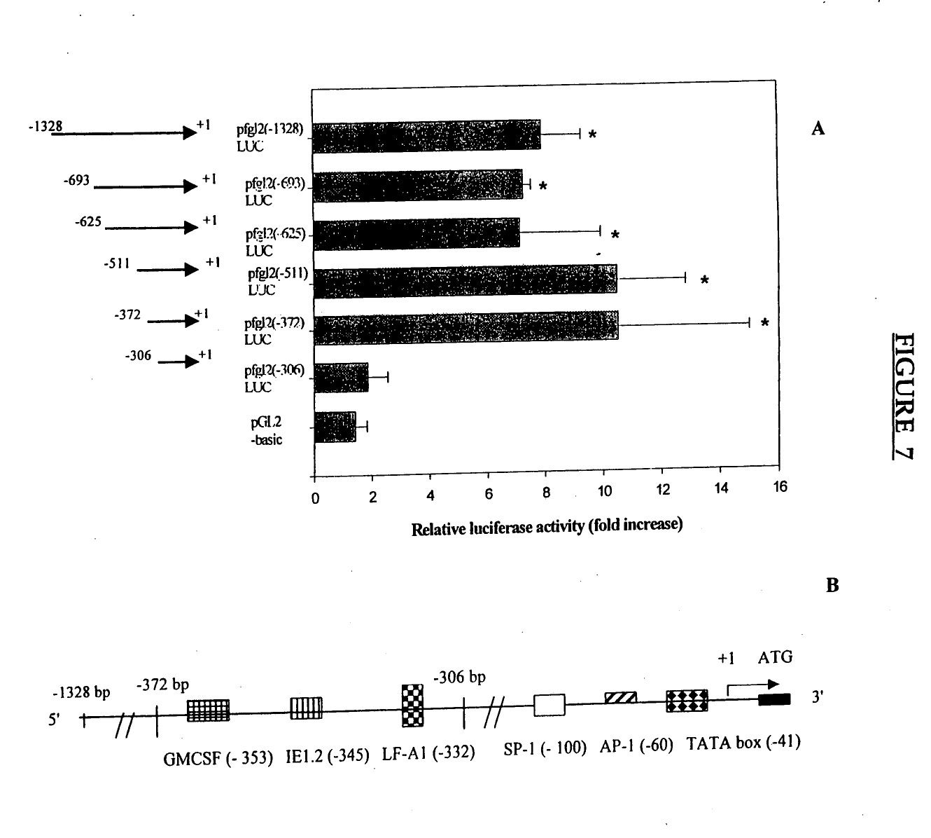

In order to characterize the region in the fgl2 promoter, which is responsive to N protein of MHV-A59, constructs containing progressive deletions of the −1328 bp fragment were cotransfected with either N gene constructs or empty pCR 3.1 vector in CHO cells (FIG. 7A). Preliminary mapping of the fgl2 promoter has defined a region from −372 to −306 to be responsive to induction of N protein. The activity of the deletion construct −306 was comparable to the promoterless plasmid pGL2-basic. Using DNAsis software, three positive cis acting regulatory elements were identified within this region which included a liver factor A1 binding element (LF-A1, −332 to −325), human cytomegalovirus immediate early gene 1.2 (IE1.2, −345 to −336) regulatory elements and granulocyte macrophage colony-stimulating factor binding element (GMCSF, −353 to −346) (FIG. 7B).

In order to determine which of the cis elements of the fgl2 promoter region were involved in N-protein induced fgl2 transcription, a series of experiments were conducted.

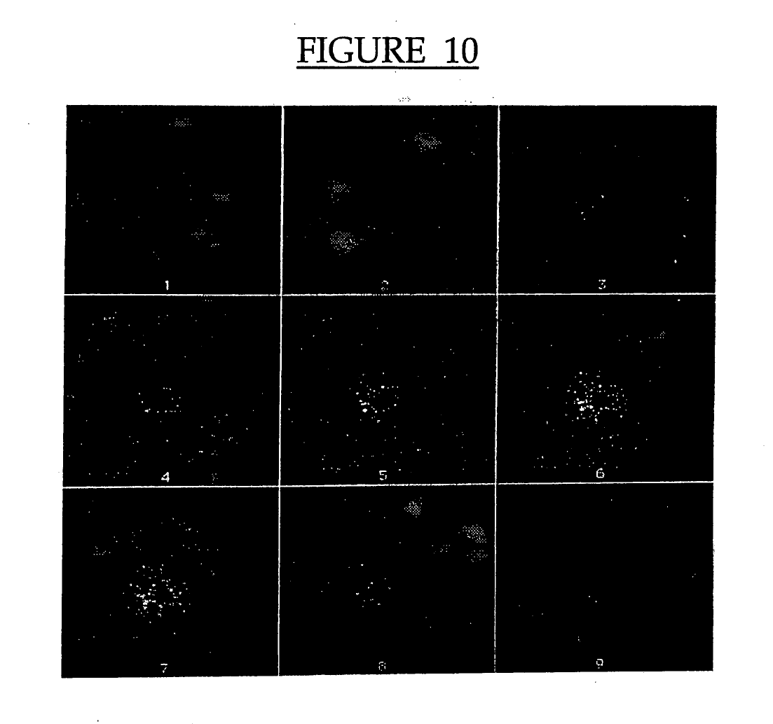

It was shown by confocus microscope immunofluoresence that n protein is present in the nucleus of infected macrophages. In FIG. 10, macrophages from Balb/cJ mice were planted as a monolayer on the glass slide flask(VWR) And infected with MHV-A59 and MHV-2. Cells were stained for nucleocapsid protein in the nucleus of infected macrophages.

The next step was to determine which of the 3 CIS elements would bind to proteins expressed in the nuclear extracts of infected cells.

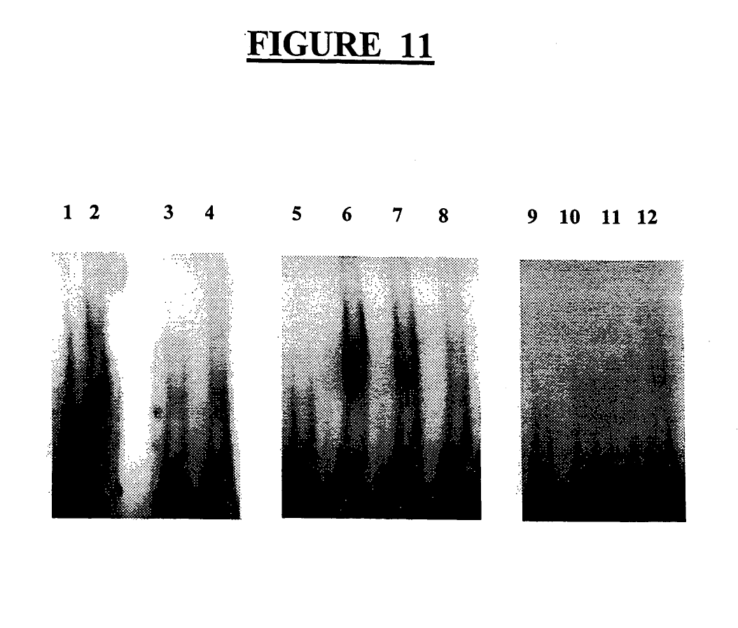

Electrophoresis mobility shift assays(EMSA) and competition studies were conducted, as shown in FIG. 11. CHO cells were incubated with MHV-259 and then DNA-protein banding was performed using band shift kit according to the instruction (Pharmacia Biotech). Band shift assays were performed with oligonucleotides corresponding to the LF-AL binding element (Lanes 1-4) or IE1.2 binding element (Lanes 5-8) or GMCSF binding element (Lanes 9-12) within the promoter region of fgl2 gene. The sequences of the oligonucleotides used were:

For the LF-A1 site (−338/−319 of fgl-2 promoter region):

5′-CAC TAG TGG ACC AAG TAT-3′ AND
5′-AAT TAT ACT TGG TCC ACT AGT G-3′;

For the IE.2 SITE (−353/−336 of fgl2 promoter region)):

5′-TTC CAA CTC TTT CCC AC-3′ AND
5′-AAT TGT GGG AAA GAG TTG CAA;

For the GMCSF site (−368/−351 of fgl2 promoter region):

5′-ACA GAC ATT TAG AGG TTC-3′ AND
5′-AAT TGA ACG TCT AAT GTC TGT-3′.

The probes used in EMSA were chemically synthesized oligonucleotides. 200 NG of sense and anti sense probe were mixed in annealing buffer in a total of 20 uL of reaction and incubated at 65° C. for 5 min, Then gradually cooled down to room temperature to be annealed to form a double strand dna probe. 2.5 uL of the double- stranded probe was labeled with 20 UCI of [(−³²P]DATP using klenow fragment (Pharmacia Biotech). DNA was separated from non-incorporated radioisotope by probe QUANT™ G50 micro-columns.

For competition experiments, nuclear extracts (nu) were preincubated with either specific competitor (cold probes) or non specific competitor. After addition of radiolabel led probes, the free DNA and the DNA protein complex were separated on a 6% POLYACRYLAMIDE GEL. Lane 1,5,9: free 32P-probe; Lane 2,6,10: 32P-probe+NU; Lane 3,8,11: 32P-probe+cold specific competitor+NU; Lane 4,7,12: 32P-probe+non specific competitor+NU.

Nuclear extracts were incubated with ³²P-labeled double-stranded oligonucleotides probe for one of the CIS ELEMENTS FOR 15 min in a total of 20 uL reaction at 4° C. In the presence of 10 MM TRIS-HCL pH 7.5, 50 MM NACL, 0.5 MM DTT, 10% GLYCEROL, 0.05% NP40, 1 UG of poly (DI-DC), 50 MM NACL and 5 MM MGCL2. Protein-DNA complexes were analyzed by electrophoresis in 5% nondenaturing gels, followed by auto radiography.

The assay results show that LF-A1 and IE1.2 oligonucleotide probes bind to protein expressed in the nuclear extracts. The banding suggests that LF-A1 binds to the LF-A1 host transcription factor of the infected cell. The LF-A1 transcription factor has previously been described in, Elizabeth M. Hardon, Monique Frain, Giacomo Paonessa and Riccardo Cortese. “Two Distinct Factors Interact With The Promoter Regions Of Several Liver-specific Genes.” The EMBO Journal Vol. 7 No.6 pp.1711-1719, 1988.

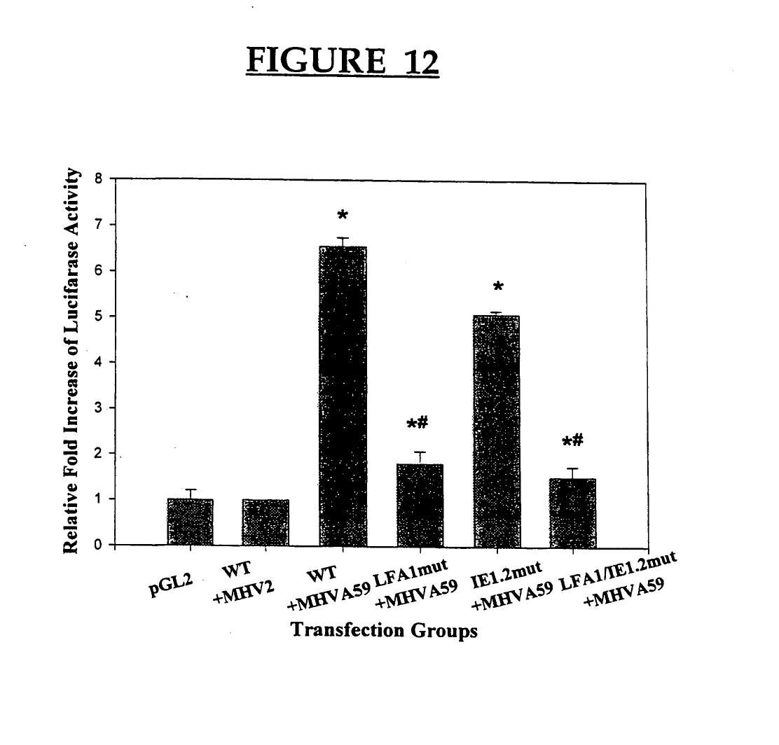

To study whether LF-A1 or IE1.2 was responsible for fgl-2 induction, the effect of mutations in these elements on transient expresion of luciferase activity of MFGL-2 was studied in response to MHV-A59 N protein in CHO cells. 0.5 Micrograms of N gene constructs from MHV-A59, MHV-2 was cotransfected with −0.25 micrograms of wild-type (WT) PFGL2 (−1328)LUC or its mutanst for candiate CIS elements LF-A1 (LF1MUT), IE1.2 (IE1.2MUT) or LFA1 and IE1.2 double mutation (LFAL/LE1.2MUT) respectively in CHO cells for 40-44 hrs. Cells were harvested and freeze-thawed three times for measurement of luciferase activity. PGL2-basic vector was used as a negative control. The results are shown in FIG. 12, where values represent the mean±S.D. of four separate experiments done in triplicate. * represents P<0.01 compared with cells cotransfected with MHV-2 N construct. # represents P<0.01 compared with cells cotransfected with wide type PFG12 (−1328) LUC. The results show that LF-A1 is responsible for fgl2 induction.

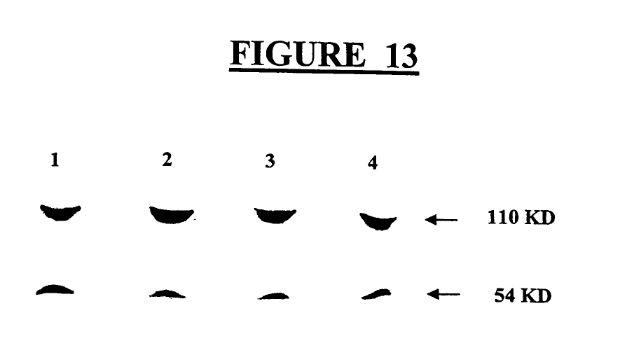

As LF-A1 has not previously been reported to be expressed in cells other than the liver and kidney, a western blot analysis for detection of LF-A1 protein in macrophages in Balb/cJ was conducted. The results are shown in FIG. 13. Nuclear proteins (NU) were extracted from macrophages infected with MHV at a multiplicity of infection (M.O.I) OF 2.5. CHO cells transfected with HNFA (LF-A1) expression construct were used as positive control. Twenty (20) ML of lysate were resolved by SDS-page and then transferred to a nitrocellulose membrane. Membrane was probed with a polyclonal antibody against the HNF-4 protein at 4° C. overnight, followed by incubation with goat anti rabbit IGG labeled with horseradish peroxidase as described under the methods and materials section above for “western blot. Lane 1. HNFA expression construct+CHO NU; Lane 2. MΦNU; Lane 3. MΦ+MHV-A59 NU; Lane 4. MΦ+MHV-2 NU. FIG. 13 shows that LF-A1 IS present in macrophages.

Summary

Studies using a model of viral hepatitis induced by infection with MHV-3 have provided significant insight into the mechanisms underlying the pathogenesis of this disease and have suggested novel approaches to therapy (Ning et al., 1998; Cattral and Levy, 1995; Sidwell et al., 1977). Furthermore, recent studies have demonstrated the role of the selective expression of the fgl2 prothrombinase in the pathogenesis of MHV-3-induced fulminant liver failure (Parr et al., 1995; Ning et al., 1998; Li et al., 1992; Ding et al., 1997).

The studies presented here demonstrate that infection of macrophages with MHV-A59 resulted in elevated PCA, a result similar to that observed during infection of macrophages with MHV-3. This contrasts with the results obtained with MHV-JHM and MHV-2, which do not induce elevations of PCA in infected macrophages. Recombinant viruses (n=5) which derive a portion of their genomic RNA from the 3′-region of MHV-A59 induces functional PCA, whereas those containing a 3′-region derived from JHM or MHV-2 (n=5) do not, suggesting that the 3′-portion of the genome may play an important role in the induction of fgl-2 transcription. The sequence differences in the genome in the 3′-area of interest lies mainly within the 5′ end of the N gene. The sequence differences in the N gene between inducers and non-inducers are a 12-nucleotide deletion at nt 111-123 of the coding region for the structural domain I and a 3-nucleotide insertion at nt 1143-1145 of the coding region for structural domain III, which may account for the inability of JHM or MHV-2 to induce fgl-2. CHO cells cotransfected with the N gene construct from A59 and with the fgl-2 promoter construct showed a 6-fold increase in luciferase activity in contrast to baseline or MHV-2-cotransfected cells.

These findings strongly suggest that the N gene is responsible for fgl-2-induction. It should be noted that induction of fgl-2 could not be explained by differences in virus replication, as MHV-2, a non-inducer, replicates to higher titers than A59 while A59 replicates to higher titers than JHM. It is also of great interest that all of the recombinants of MHV-2×A59 have the MHV-2-derived leader sequence whereas the majority of recombinants between A59×JHM contain the A59 leader (Keck et al., 1988). It is not clear whether the leader sequence differences among viruses are responsible for the altered growth property of the virus.

MHV N protein has been proposed to consists of three conserved structural domains (I basic, II basic, and III acidic) which are tethered to each other by two regions of variable amino acid composition (designated A and B) (Parler, M. M. and Masters, P. S., 1990). The sequence differences in the N gene between viruses which induce fgl-2, such as MHV-3 and MHV-A59, and MHV-2 lies within two regions corresponding to domain I (nt 400-500) and domain III (nt 1100-1200), respectively. The inventor has shown that it is primarily the diferences in domain I which account for inability of MHV-2 to induce the fgl2 gene.

Co-transfection of an N gene expression construct with the fgl2 promoter/reporter luciferase confirms that the N gene of A59 accounts for induction of fgl2. Possible mechanisms by which n protein induces fgl2 prothrombinase expression include the transport of the N protein into the nucleus of infected cells, acting as a transcription activator for fgl2; or modulation of signal transduction pathways that regulate host transcription machinery, thereby increasing the steady state levels for fgl2 transcriptsan alternative mechanism may be N protein binding directly to fgl2 mrna, thus altering the rate of fgl2 transcript degradation.

The inventors have now shown that the mechanism by which N protein induces fgl2 prothrombinase expression most likely includes the phosphorylation of LF-A1, transport of the LF-A1 protein into the nucleus of infected cells, acting to modulate the signal transduction pathways that regulate host transcription machinery, thereby increasing the steady state levels for fgl2 transcripts. Recent work by both nuclear runoff assays and transient transfection experiments have demonstrated that the induction of fgl2 mRNA by MHV-3 infection is at least in part attributable to new transcription (Leibowitz, J. L. et al., in press).

Preliminary mapping of the fgl2 promoter has defined a region from −372 to −306 to be responsive to the N protein. The present inventors identified three putative cis-elements and have shown that LF-gibinding element is the CIS element necessary for FGL-2 transcription. It was also shown that lf-ai is present in macrophages. Previous reports which have suggested that LF-A1 was typical of a liver specific gene and liver-enriched transcription factor. Furthermore, LF-A1 is known to regulate gene expression of coagulation factors IX, X and XII (Crossley et al., 1992; Citarella et al., 1993; Miao et al., 1992). The present data shows that GMCSF is not invovled in ainducing fgl-2, however, IE1.2 may play a minor role. IE1.2 has been implicated as human cytomagelovirus immediate early gene 1 and 2 regulatory elements, providing a link between virus infection and inflammation (Fickenscher et al., 1989).

Recent studies have shown that the severity of hepatic injury in patients with hepatitis B is related to the synthesis and expression of nucleocapsid protein of HBV, suggesting that the accumulation of hepatitis B core antigen may damage hepatocytes directly or may serve to stimulate cell mediated immune responses (Davies, S. E. et al., 1991; Chisar, F. V. and Ferrari, C., 1995). The inventor has recently cloned and sequenced the human prothrombinase gene (hfgl2) gene and have shown its expression in the liver of 3 patients with fulminant hepatic failure). It is expected that the hepatitis B core antigen induces transcription of hfgl2.

In conclusion, mapping of genetic determinants in parenteral and recombinant MHV strains demonstrates that the N protein of strains of MHV which induce FHF is responsible for enhanced transcription of the fgl-2 prothrombinase gene. These studies provide significant insights into the viral pathogenesis of human diseases such as hepatitis B and C in which core antigen or pathogenic factor (nucleocapsid protein) influences disease activity.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

Full Citations for References Referred to in the Specification

1. Cattral, M. C. and G. A. Levy. 1995. Artificial liver support \overline{n} pipe dream or reality. N. Engl. J. Med. 331:268-69.

2. Chisari, F. V. and C. Ferrari. 1995. Hepatitis B virus immunopathology. Springer Semin. Immunopathol. 17:261-281.

3. Citarella, F., Misiti, S., Felici. A., Aiuti, A., Porta, C. L., Fantoni, A. (1993). Biochim Biophys Acta. 1172, 197-199.

4. Crossley, M., Ludwig, M., Stowell, K. M., Vos, P. D., Olek, K., Brownlee, G. G. (1992). Science. 257, 377-379

5. Davies, S. E., B. C. Portmann, J. G. Grady, P. M. Aldis, K. Chaggar. 1991. Hepatic histologic findings after transplantation for chronic hepatitis B virus infection, including a unique pattenpattern of fibrosing cholestatic hepatitis. Hepatology 13: 150-157.

6. Ding, J. W., Q., Ning, M., Liu, K. M., Peltekian, L. S., Fung, C., Holloway, P. A., Marsden, H., Yeger, E. H., Cole, M. J., Phillips, G. A. Levy. 1997. Fulminant hepatic failure in murine hepatitis virus strain 3 infection: tissue-specific expression of a novel fgl2 prothrombinase. J. Virol. 71:9223-9230.

7. Evans, R., and S. J. Kamdar. 1990. Stability of RNA isolated from macrophages depends on the removal of an RNA-degrading activity early in the extraction procedure. Biotechniques. 8:357-360.

8. Fickenscher, H., Stamminger, T., Ruger, R., Fleckenstein, B. (1989). J Gen Virol. 70, 107-123.

9. Gasson, J. C. (1991). Blood. 77, 1131-1145.

10. Keck, H. J. G., S. A., Stohlman, L. H., Soe, S., Makino, M. M. C. Lai. 1997. Multiple recombination sites at 5′-end of murine Coronavirus RNA. Virology 156:331-343.

11. Keck, J. G., L. H. Soe, S. Makino, S. A. Stohlman, M. M. Lai. 1988. RNA recombination of murine Coronaviruses: recombination between fusion-positive mouse hepatitis virus A59 and fusion-negative mouse hepatitis virus 2 . J. Virol. 62:1989-1998.

12. Koyama, T., Hall, L. R., Haser, H. G., Tonegawa, S., Saito, H. (1987). Proc. Natl. Acad. Sci. USA. 84, 1609-1613.

13. Lai, M. M. C. and S. A. Stohlman. 1978. RNA of mouse hepatitis virus. J. Virol. 26: 236-242.

14. Lai, M. M. C. and D. Cavanagh. 1997. The molecular biology of Coronaviruses. Adv. Virus Res. 48:1-100.

15. Lai, M. M. C. RNA recombination in animal and plant viruses. 1992. Microbiol. Rev. 56:61-79.

16. Lavi, E., E. M., Murray, S., Makino, S. A., Stohlman, M. M., Lai, S. R. Weiss. 1990. Determinants of Coronavirus MHV pathogenesis are localized to 3′ portions of the genome as determined by ribonucleic acid-ribonucleic acid recombination. Lab. Invest. 62:570-578.

17. Leibowitz, J. L., M. F. Liu, B. Pereira, M. Belyavskyi, P. Marsden, G. Levy. Transcriptional and sequencing studies of the fgl2 gene representing the mouse hepatitis virus, strain 3 (MHV-3), induced macrophages prothrombinase. Genomics, in press.

18. Levy, G. A., J. L., Leibowitz, T. S. Edgington. 1981. Induction of monocyte procoagulant activity by murine hepatitis virus type 3 parallels disease susceptibility in mice. J. Exp. Med. 154:1150-1163.

19. Levy, G. A., P., Macphee, L. S., Fung, M. M., Fisher. and A. M. Rappaport. 1983. The effect of mouse hepatitis virus infection on the microcirculation of the liver. Hepatology 3:964-973.

20. Li, C., L. S., Fung, A., Crow, N., Myers-Mason, J., Leibowitz, E., Cole, G. Levy. 1992. Monoclonal antiprothrombinase A(3D4.3) prevents mortality from murine hepatitis virus (MHV-3) infection. J. Exp. Med. 176:689-697.

21. Mackman, N., Fowler, B. J., Edgington, T. S., Morrissey, J. H. (1990). Proc Natl Acad Sci USA. 87, 2254-2258.

22. Macphee, P. J., V. J., Dindzans, L. S., Fung and G. A. Levy. 1985. Acute and chronic changes in the microcirculation of the liver in inbred strains of mice following infection with mouse hepatitis virus type 3. Hepatology 5:649-660.

23. Makino, S, J. O., Fleming, J. G., Keck, S. A., Stohlman, and M. M. C. Lai. 1989. RNA recombination of Coronaviruses: Localization of neutralizing epitopes and neuropathogenic determinants on the carboxyl terminus of peplomer. Proc. Natíl. Acad. Sci. USA. 84:6569-6571 (1989)

24. Makino, S., J. G., Keck, S. A., Stohlman, M. M. C. Lai. 1986. High-frequency RNA recombination of murine Coronaviruses. J. Virol. 57:729-737.

25. Masters, P. S. and L. S. Sturman. 1990. Functions of the Coronavirus nucleocapsid protein. Coronaviruses and their diseases. Academic Press, New York, 235-238.

26. Miao, C. H., Leytus, S. P., Chung, D. W., Davie, E. W. (1992). J. Biol. Chem. 267, 7395-7401.

27. Ning, Q., D., Brown, J., Parodo, M., Cattral, R., Gorczynski, E., Cole, L., Fung, J. W., Ding, M. F., Liu, O., Rotstein, M. J., Phillips., G. Levy. 1998. Ribavirin inhibits viral-induced macrophage prodution of TNF, IL-1, the procoagulant fgl2 prothrombinase and preserves Th1 cytokine production but inhibits Th2 cytokine response. J. Immunol. 160:3487-3493.

28. Pachuk, C. J., P. J., Bredenbeek, P. W., Zoltick, W. J. W., Spaan, S. R. Weiss. 1989. Molecular cloning of the gene encoding the putative polymerase of mouse hepatitis Coronavirus, strain A59. Virology 171:141-152.

29. Parler, M. M. and P. S. Masters. 1990. Sequence comparison of the N genes of five strains of the Coronavirus mouse hepatitis virus suggests a three domain structure for the nucleocapsid protein. Virology 179:463-468.

30. Parr, R. L., J. L., Leibowitz, L. S., Fung, S. J., Reneker, N., Myers-Mason, G. A. Levy. 1995. Association of mouse fibrinogen-like protein with murine hepatitis virus-induced prothrombinase activity. J. Virol. 69:5033-5038.

31. Russell, N. H. (1992). Blood Rev. 6, 149-156.

32. Sidwell, R. W., J. H., Huffman, N., Campbell, L. B., Allen. 1977. Effect of ribavirin on viral hepatitis in laboratory animals. Ann.NY. Acad. Sci. 284:239-246.

### DETAILED FIGURE LEGENDS

FIG. 1. The schematic representation of oligonucleotide maps of the recombinant viruses (MHV-A59×JHM) and (MHV-A59×MHV-2). L: leader sequence. Pol: RNA dependent RNA polymerase gene. HN: hemagglutinin-esterase glycoprotein gene. S: spike protein. M: matrix glycoprotein gene. N: nucleocapsid protein gene. The data are from Parler, M. M. and P. S. Masters, 1990.

FIG. 2. A schematic diagram of the organization of the fgl2 gene is shown in the upper panel. The lower panel shows the sequence of the 1.3 kb DNA flanking 5′ end of fgl2. The sequence has been deposited into Genbank with accession number AF025817. The sequence shows the overlapping 400 bp at 3′ end of this fragment and the 5′ end of the published sequence by Koyama et. al (Koyama et al., 1987) with the accession number M15761 are consistent. The putative cis-elements responsive to N protein and initiating ATG for translation are indicated in bold and underlined.

FIG. 3. Expression of Balb/cJ macrophage functional PCA induced by parental viruses and their recombinants derived from MHV-A59×JHM (A) or MHV-A59×MHV-2 (B).

Macrophages from Balb/cJ were infected with viruses at a multiplicity of infection (MOI) of 2.5 for 8-10 h and harvested for measurement of PCA activity. Values represent the mean±SD of three separate experiments done in triplicate. * represents p<0.01 when compared with unstimulated macrophages.

FIG. 4. Expression of Balb/cJ macrophage fgl2 transcripts induced by parental viruses and their recombinants by RT-PCR. 5 μg of total cellular RNA extracted from infected macrophages was reverse transcribed and then PCR was performed using specific fgl2 primers as described in Materials and Methods. Lane 1, macrophages+MHV-3; Lane 2, macrophages+MHV-A59; Lane 3, macrophages+MHV-JHM; Lane 4, macrophages+MHV-2; Lane 5, macrophages+MHV-RL1; Lane 6, macrophages+MHV-CA13; Lane 7, macrophages+MHV-CA43; Lane 8, macrophages+MHV-ML3; Lane 9, macrophages+MHV-ML9; Lane 10, macrophages+MHV-ML11. PCA of glyceraldehyde-3-phosphate dehydrogenase (GAPDH) was set up as a technique control.

FIG. 5. Expression of nucleocapsid (N) protein in transfected cells by Western blot analysis. 2×106 of cell lysis post transfection were loaded in a 10% of SDS-PAGE gel and transferred to a nitrocellulose membrane. Membrane were then probed with antibodies as described in Materials and Methods. Lane 1, CHO+fgl2 promoter construct+A59 N gene construct; Lane 2, CHO+fgl2 promoter construct+MHV-2 N gene construct; Lane 3, CHO+fgl2 promoter construct+pCR 3.1 vector alone; Lane 4. 17 CL1 cells+MHV-3.

FIG. 6. Effect of N protein on fgl2 promoter. 0.5 μg of N gene construct were cotransfected with 0.5 μg of fgl2 promoter construct in CHO cells for 40-44 h and cells were harvested and freeze-thawed 3 times for measurement of luciferase activity. Values represent the mean±SD of five separate experiments done in triplicate. * represents p<0.01 when compared with cells cotransfected with pCR 3.1 vector.

FIG. 7. A. Transient expression of luciferase activity by deletion constructs of the fgl2 promoter in response to MHV-A59 N protein in CHO cells. A series of the fgl2 promoter constructs containing varying lengths of the fgl2 promoter sequence were cotransfected with a MHV-A59 N gene construct into CHO cells. Relative luciferase activity is expressed in fold increase compared to CHO cells cotransfected with the fgl2 promoter constructs with empty pCR3.1 vector. PGL2-basic vector was used as a negative control. All luciferase assays represent the mean SD of six or more independent experiments. * represents p<0.01 when compared with cells cotransfected with empty pCR 3.1 vector.

B. Schematic representation of the putative regulatory elements in the putative (−372 to −306) fgl2 promoter responsive to N protein. Also shown are the ATG translation initiation site and the location of the TATA box.

FIG. 8. Expression of Balb/cJ macrophage functional PCA induced by MHV and it I mutant. Macrophages from Balb/cJ were infected with MHV and MHV-A59 I mutant (Alb 110 and it isogenic wide type Alb 111) at a M.O.I. of 2d.5 for 8-10 hrs and harvested for measurement of PCA activity. Values represent the mean±S.D. of three separate experiments done in triplicate. * represents p<0.01 compared with unstimulated macrophages.

FIG. 9. Transient expression of luciferase activity by mfgl2 promoter in response to MHV-A59 N protein and it mutants in CHO cells. N gene constructs from MHV-A59 and MHV-2 and a series of N gene mutants from MHV-A59 was cotransfected with the wide type fgl2 promoter respectively into CHO cells. Relative luciferase activity is expressed in fold increase compared with CHO cells cotransfected with N construct from MHV-2. PGL2-basic vector was used as a negative control. Values represent the mean±S.D. of five separate experiments done in triplicate. * represents p<0.01 compared with cells cotransfected with MHV-2 N construct. # represents p<0.01 compared with cells cotransfected with N gene construct from wide type MHV-A59.

FIG. 10. Confocus Microscope Immunofluoresence for detection of N protein. Macrophages from Balb/cJ mice were planted as a monolayer on the glass slide flask

(VWR) and infected with MHV-A59 and MHV-2. Cells were stained for nucleocapsid protein in the nucleus of infected macrophages.

FIG. 11. Electrophoresis mobility shift assays (EMSA) and competition studies. Band shift assays were performed with oligonucleotides corresponding to the LF-A1 (lanes 1-4) or IE1.2 (lanes 5-8) or GMCSF (lanes 9-12) binding sites within the promoter region of fgl2 gene. For competition experiments, nuclear extracts (Nu) were preincubated with either specific competitor (cold probes) or non specific competitor. After addition of radiolabel led probes, the free DNA and the DNA protein complex were separated on a 6% polyacrylamide gel. The sequences of the oligonucleotides used are shown in the description. Lane 1,5,9: Free 32P-probe; lane 2,6,10: 32P-probe+Nu; lane 3,8,11: 32P-probe+cold specific competitor+Nu; lane 4,7,12: 32P-probe+non specific competitor+Nu.

FIG. 12. Transient expression of luciferase activity by mfgl2 promoter and its mutants for candidate cis-elements in response to MHV-A59 N protein in CHO cells. 0.5 ug of N gene constructs from MHV-A59, MHV-2 was cotransfected with 0.25 ug of wildtype (WT) pfgl2 (−1328) LUC or its mutants for candidate cis elements LF-A1 (LFA1mut), IE1.2 (IEI1.2mut) or LFA1 and IE1.2 double mutation (LFA1/lE1.2mut) respectively in CHO cells for 40-44 hrs, cells were harvested and freeze-thawed three time for measurement of luciferase activity. PGL2-basic vector was used as a negative control. Values represent the mean±S.D. of four separate experiments done in triplicate. * represents p<0.01 compared with cells cotransfected with MHV-2 N construct. # represents p<0.01 compared with cells cotransfected with wide type pfgl2 (−1328) LUC.

FIG. 13. Western blot analysis for detection of LF-A1 protein in macrophages in Balb/cJ. Nuclear proteins (Nu) were extracted from macrophages infected with MHV at a multiplicity of infection (M.O.I) of 2.5. CHO cells transfected with HNF-4 expression construct were used as positive control. Twenty (20) μl of lysate were resolved by SDS-PAGE and then transferred to a nitrocellulose membrane. Membrane was probed with a polyclonal antibody against the HNFA protein at 4° C. overnight, followed by incubation with goat anti rabbit IgG labeled with horseradish peroxidase as described in the Materials and Methods of the description. Lane 1. HNF-4 expression construct+CHO Nu; lane 2. My Nu; lane 3. Mφ+MHV-A59 Nu; lane 4. Mφ+MHV-2 Nu.

TABLE 1
Identity comparison of N gene sequence

| | M35156 | MHV-2 |
|---|---|---|
| | (1.666 Kb) | (1.657 Kb) |
| MHV-A59 (1.666 Kb) | 99% | 93% |
| MHV-ML3 (1.666 Kb) | 99% | 92% |
| MHV-ML11 (1.657 Kb) | 93% | 99% |

Multiple clones from each construct were sequenced. T7 primer and pCR3.1 reverse primer were used for 5′ to 3′ and 3′ to 5′ sequence, respectively. A new primer at position 557 was also designed to accomplish the cloned entire N gene sequence as described in Materials and Methods. The sequence was analyzed using the DNAsis for windows, sequence analysis software.

We claim:

1. A method of preventing or reducing immune coagulation caused by a virus comprising administering an effective amount of an inhibitor of a nucleocapsid gene (N-gene) or protein (N-protein) to an animal in need thereof.

2. A method according to claim 1 wherein the virus is a hepatitis virus.

3. A method according to claim 2 wherein the hepatitis virus is capable of inducing fgl-2.

4. A method according to claim 1 wherein the inhibitor of the N-protein is an antibody that binds to the N-protein or domain I of the N-protein.

5. A method according to claim 1 wherein the inhibitor of the N-gene is an antisense oligonucleotide that is complimentary to a nucleic acid sequence from the N-gene.

6. A method of inducing immune coagulation comprising administering an effective amount of a N-protein, domain I of the N-protein, or gene to an animal in need thereof.

7. A composition for use in inhibiting procoagulant activity in an animal comprising (a) an antibody specific for a N-protein or domain I of the N-protein; (b) or antisense nucleic acid molecules complimentary to the N-protein gene.

8. A composition for use in inducing procoagulant activity in an animal comprising a nucleic acid sequence encoding N-protein or domain I of N-protein; or an N-protein or domain 1 of N-protein in admixture with a suitable diluent or carrier.

9. A vaccine against a virus causing immune coagulation comprising an amount of an N-protein or peptide which is effective to induce an immune response against N-protein.

10. A vaccine for treating or preventing viral hepatitis comprising an effective amount of an N-protein or peptide in admixture with a suitable diluent or carrier.

11. A method of preventing or reducing immune coagulation caused by a virus comprising administering an effective amount of an inhibitor of LF-A1 gene or protein to an animal in need thereof.

12. A method according to claim 11 wherein the virus is a hepatitis virus.

13. A method according to claim 11 wherein the inhibitor of the LF-A1 protein is an antibody that binds to the LF-A1 protein.

14. A method according to claim 1 wherein the inhibitor of LF-A1 protein is an antisense oligonucleotide that is complimentary to the LF-A1 binding element of the promoter region of the fgl-2 gene.

15. A method according to claim 11 wherein the inhibitor of the LF-A1 gene is an antisense oligonucleotide that is complimentary to a nucleic acid sequence from the LF-A1 gene.

16. A method of inducing immune coagulation comprising administering an effective amount of a LF-A1 protein or gene to an animal in need thereof.

17. A composition for use in inhibiting procoagulant activity in an animal comprising (a) an antibody specific for a LF-AL protein; (b) or antisense nucleic acid molecules complimentary to the LF-A1 protein gene; or (c) antisense nucleic acid molecules complimentary to the LF-A1 binding element of the promoter region of the fgl-2 gene.

18. A composition for use in inducing procoagulant activity in an animal comprising a nucleic acid sequence encoding LF-A1 protein or an LF-A1 protein in admixture with a suitable diluent or carrier.
